(19) United States
(12) Patent Application Publication
(10) Pub. No.: US 2008/0039708 A1
(43) Pub. Date: Feb. 14, 2008

(54) Apparatus and method for assessing body composition

(75) Inventors: Gersh Z. Taicher, Houston, TX (US); Arcady Reiderman, Houston, TX (US); Israel Kovner, Houston, TX (US); Zinovy Krugliak, Houston, TX (US)

Correspondence Address: RICHARD A. FAGIN, P.O. BOX 1247, RICHMOND, TX, 77406-1247, US

(73) Assignee: ECHO MEDICAL SYSTEMS, L.L.C.

(21) Appl. No.: 11/502,675

(22) Filed: Aug. 10, 2006

(51) Int. Cl. A61B 5/05

(52) U.S. Cl. 600/410

(57) ABSTRACT

A method for analyzing composition of a human body includes inducing a substantially homogeneous static magnetic field in the entire body. A substantially homogeneous radio frequency magnetic field is induced in the entire body so as to induce nuclear magnetic resonance effects in the body. Nuclear magnetic resonance signals emanating from the entire body are analyzed to determine body composition.

### CROSS-REFERENCE TO RELATED APPLICATIONS

Not applicable.

### STATEMENT REGARDING FEDERALLY SPONSORED RESEARCH OR DEVELOPMENT

Not applicable.

### BACKGROUND OF THE INVENTION

1. Field of the Invention

The invention is related to the field of Nuclear Magnetic Resonance (“NMR”) and Magnetic Resonance Imaging (“MRI”) apparatus and methods. More particularly, the invention relates to apparatus and methods for determining a known component from a mixture of unknown components. More specifically, the invention relates to methods and apparatus for using NMR for precise and quantitative determination of body composition in Humans. In one application methods and apparatus according to the invention relate to using NMR for rapid, quantitative in-vivo determination of tissue properties, such as Fat Mass and Lean Mass. In another application methods and apparatus according to the invention relate to using NMR for rapid, quantitative in-vivo determination of therapeutic outcome of drug or nutritional intervention.

2. Background Art

The description of the invention and its background are explained herein in the context of Fat Mass and Lean Mass determination. It is to be explicitly understood, however, that the invention is not limited to analysis and monitoring of Fat Mass and Lean Mass. For example, Fat Mass, Lean Mass and Bone Mass may also be determined using methods and apparatus according to the invention. Fat composition (different fatty acids), lean composition (water, protein, and glycogen), and bone composition (mineral, collagen, and water) may also be determined using methods and apparatus according to the invention.

In human health monitoring and treatment, the level of total body mass that is derived from adipose mass is the variable that has been determined empirically to be most closely associated with risk for pathology. Advanced models of body composition and newer technologies that precisely and accurately calculate adipose mass may eventually replace simple anthropometric methods such as body weight, height, waist circumference, skin fold thickness, etc. in determining likelihood of pathology.

Body Mass Index (“BMI”) is defined as body weight (kg)/height² (m²). Although BMI is a reasonable marker of energy balance for individuals, it is very rough marker of adiposity across populations.

Hydrostatic weighing or Under Water Weighing (“UWW”) has been the most preferred technique for human whole body composition analysis for several decades. However, due to several practical inconveniences and questionable underlying assumption its usage is limited. UWW assess whole body fat content expressed as a percentage of body weight. See, for example, U.S. Pat. No. 4,873,866 to Fairbanks.

UWW based on a two-component (2C) body composition model assumes specific densities 0.9 and 1.1 g/cm³ for Fat Mass (FM) and Fat-Free Mass (FFM) respectively. UWW further assumes that these densities are constant within different individuals or populations. Whole body densities have been determined to vary in a range between 1.08 g/cm³ (very lean) and 1.00 g/cm³ (severely obese).

Other UWW techniques are based on four-component (4C) or three-component (3C) body composition models. 4C and 3C models additionally use assumptions that FM is composed of constant proportions of water (73.2%), minerals (6.8%), and protein (19.5%) each having a specific density assumed to be constant at body temperature. Precise measurement of Total Body Water (TBW) and Bone Mineral Content (BMC) are required to use 4C and 3C models because of the potential for additional error in the final results for FM that is related to TBW and BMC measurements. In certain human population groups, such as children, the elderly, African-Americans, or sick patients, 4C or 3C methods may provide more accurate estimates of FM than the 2C method.

UWW is not practical for accurate measurements in individuals having cardiovascular or pulmonary disorders, elderly, young children, and very obese subjects. Substantial errors may occur due to body movement and the buoyant effects of air in the gastrointestinal tract and lungs. The simultaneous measurement of residual lung volume and underwater weight may be preferred because it controls for the effects of the increased pressure of water on the thorax during immersion. Inaccurate measurements of air in the lungs can be a major source of error when estimating body density from underwater weighing. However, UWW may be the only practical method of measuring body fat in very obese subjects who cannot be evaluated by other methods.

U.S. Pat. No. 4,144,763 to Vogelman and U.S. Pat. No. 5,105,825 to Dempster disclose plethysmography apparatuses and methods. Plethysmography is a more convenient way for measuring body adiposity as compared to UWW. Measurement of body density by plethysmography allows for a high degree of precision in volume measurement, but inconsistencies in body density, the necessity for lung volume correction, variation in skeletal mass, and degree of hydration are not accounted for by plethysmography methods.

U.S. Pat. No. 6,393,317 to Fukuda et al. and U.S. Pat. No. 5,415,176 to Sato et al. disclose two examples of widely used techniques for fat assessment based on body bioelectrical impedance. A method for fat assessment based on body electrical conductivity is described by Unangst E. T., Jr., and Merkley L. A. in, The effects of lipid location on non-invasive estimates of body composition using EM-SCAN technology, J. Exp. Biol., 2002:205 (Pt. 19) pp. 3101-3105.

None of the foregoing methods of body composition analysis have been broadly implemented, largely because of inaccuracy and poor specificity of the results. Measurement of body composition of experimental animals by plethysmography, hydrostatic weighing (“UWW”), bioelectrical impedance, and electrical conductivity has not proven to be practical.

In order to provide a more precise quantitative measure of whole body composition in animals and humans, the Dual Energy X-ray Absorptiometry (“DEXA”) technique is more widely used than the foregoing techniques. U.S. Pat. No. 6,233,473 to Shepherd et al. discloses a method of body composition analysis using a dual-energy, fan-shaped distribution of X-rays, and detector signal processing that corrects for mass magnification and other effects due to the geometry of the measurement system. In the method disclosed in the '473 patent, the thickness of the attenuating material along respective ray paths is obtained by using a four-dimensional look-up table derived experimentally from step-wedge measurements, and another look-up table and interpolation between table entries are used to convert projected mass to true mass.

DEXA precision differs with the instrument type, the particular animal species being evaluated, the software and the actual methods that are used. The basic physical principle of DEXA is associated with attenuation of X-rays transmitted through an object. The degree of attenuation (attenuation coefficient) depends on the object's thickness, density, and chemical composition as well as the initial energy of the X-ray photons. At low initial photon energies (less than about 0.8 million electron volts), photon attenuation is non-linear, and is governed by the photoelectric effect and by Compton scattering. If the object under evaluation is composed of two or more homogeneous materials, then the composite attenuation coefficient may be approximated by a weighted sum of the individual attenuation coefficients, each weighted for its fractional contribution to the total mass.

The attenuation of X-rays through lean human body tissue and fat tissue is slightly different, but is substantially different for bone tissue, primarily because of their differences in density and chemical composition. DEXA does not provide three independent measurements, even though three body composition values: bone; lean; and fat tissue fractional amounts are reported. With increasing initial photon energy, the differences in the attenuation properties for these three types of body tissue decrease.

The following is summary of a DEXA technique for whole body composition analysis of animals and humans. First, a record is made of the attenuation of X-rays at both initial photon energy values in air. Then the pixel size, scanning speed and beam size are selected. A scan of the object (mouse) is then made. The detected X-ray photon amplitudes and count rates are corrected for detector dead time loss, spill-over from one energy window to another, and for beam hardening. From two equations (two photon energy levels) the amount of soft tissue and bone mineral is then determined.

Soft tissue in the non-bone pixels is separated into fat and lean mass by means of a calibration that translates attenuation coefficients into fat fractions. Corrections are made for tissue thickness variation. The fat content of the soft tissue layer overlying, underlying and/or inside bone is estimated based on predetermined relationships between fat-to-lean ratio of pure soft tissue surrounding bone.

The main advantage of DEXA is the ability to analyze individual regions within an entire body. DEXA as a method for analyzing whole body composition may be subject to the following limitations. First is the assumption that the composition of the soft tissue layer overlying bone has the same Fat-to-Lean Ratio, or the ration is related in a predetermined way to the Fat-to-Lean ratio of other non-bone tissues. For a whole body scan, about 40% of the pixels are typically classified as containing bone. Next, thicker tissue regions remove more low energy photons from the radiation beam as compared to thinner regions, this effect being known as “beam hardening.” Further, DEXA assumes homogeneous hydration of lean tissues.

In the field of in-vivo analysis of body composition parameters there have been numerous attempts to use nuclear magnetic resonance (“NMR”) methods and apparatus. Briefly, these techniques and their limitations are as follows.

I. Magnetic Resonance Spectroscopy (“MRS”). The MRS method used to quantify fat content in a body is based on recording a ¹H (proton) spectrum in-vivo. An example of using a standard MRS apparatus for such analysis is described by Mystkowski et al. in, Validation of whole-body magnetic resonance spectroscopy as a tool to assess murine body composition”, Int. J. of Obesity, 2000:24, pp. 719-724. A drawback to the technique disclosed in the Mystkowski et al. paper is the fact that many human tissue types contain a variety of lipids which yield ¹H spectral peaks within a very narrow chemical shift range. In addition, MRS requires very high homogeneity and strength of the static magnetic field, due to the required high spectral resolution of chemical shifts, making MRS equipment extremely expensive.

II. Magnetic Resonance Imaging (“MRI”). A MRI method for body composition analysis is described by Ross et al. in, Quantification of adipose tissue by MRI: relationship with anthropometric variables, J. Appl. Physiol. 1992:72(2) pp. 787-795, and in U.S. Pat. Nos. 5,225,781; 5,594,336; 6,147,492; and 5,644,232. MRI equipment is expensive and does not provide accurate analysis results due to effects of motion of the patient being examined, inhomogeneity of the sensitivity function and interpolation error between acquired 2D images in transverse image slices.

III. NMR Relaxometry. NMR relaxometry methods known in the art avoid the necessity for complicated and expensive equipment. NMR relaxometry methods known in the art, however, have several limitations, such as with respect to accuracy and precision. Kamman et al., Multi-exponential relaxation analysis with MR imaging and NMR spectroscopy using fat-water systems, Magn. Reson. Imaging 1987:5(5) pp. 381-392 describes a NMR relaxometry method for body composition analysis.

Despite extensive research and development into methods of whole body composition analysis, there is still a need for reliable, accurate, precise, and specific non-invasive methods for acquiring information relating to body fat mass, lean mass, total water content, etc. In particular, it is a purpose of the present invention to make acquired NMR signal equally sensitive to all different region of the body, so repositioning of the body or its motion during the measurement substantially do not affect to the precision of the measurements. It is another purpose of the present invention is to develop a method and inexpensive equipment for fast, high precision measurement of the whole body composition of large live objects like adult humans.

### SUMMARY OF THE INVENTION

One aspect of the invention is a method for analyzing composition of a human body. A method according to this aspect of the invention includes inducing a substantially homogeneous static magnetic field in the entire body, inducing a substantially homogeneous radio frequency magnetic field in the entire body so as to induce nuclear magnetic resonance effects in the body, and analyzing nuclear magnetic resonance signals emanating from the body.

An apparatus for analyzing composition of a human body according to another aspect of the invention includes a magnet for inducing a substantially homogeneous static magnetic field in a chamber having a volume at least as large as an entire human body. The apparatus includes means for inducing a substantially homogeneous, pulsed radio frequency magnetic field in the entire human body and means for analyzing nuclear magnetic resonance signals from the entire body induced therein by the static magnetic field and the radio frequency magnetic field.

Other aspects and advantages of the invention will be apparent from the following description and claims.

### BRIEF DESCRIPTION OF THE DRAWINGS

FIG. 1 shows one embodiment of a NMR apparatus according to the invention.

FIG. 2A shows one embodiment of an antenna according to the invention.

FIG. 2B shows a graph of RF magnetic field amplitude with respect to axial position along the example apparatus shown in FIG. 1.

FIG. 2C illustrates possibility of movement of an object being examined by the apparatus of FIG. 1 without materially affecting measurements made by the apparatus.

FIG. 3A is a graph of frequency content of different types RF pulses applied to the antenna of the apparatus of FIG. 1.

FIG. 3B is a graph of static magnetic field amplitude with respect to axial position along a sample chamber of the apparatus of FIG. 1, and corresponding magnetic resonance conditions with respect to the RF pulse frequency of FIG. 3A.

FIG. 3C is a graph of relative sensitivity of NMR measurement with respect to position within the sample chamber for various RF pulse types as shown in FIG. 3A.

FIG. 4 shows modeling results for a nuclear magnetization spectrum illustrating the effect of frequency jittering.

FIG. 5 shows an example of inhomogeneous nuclear magnetization being corrected using correcting filter in the receiver

FIG. 6 shows NMR signal spectrum shift due to time instability of the static magnetic field.

FIG. 7 is a graph of signal to noise with respect to sample chamber volume and static magnetic field amplitude.

FIG. 8 shows a magnet assembly used for whole body composition measurements in humans.

FIG. 9 is a view from an end of the magnet/antenna assembly showing RF shielding and an active RF dipole spoiler.

FIG. 9A shows a particular implementation of RF antenna.

FIG. 10 represents NMR relaxation curves for fat tissue, lean tissue and free water at 250 KHz.

FIG. 11 illustrates results of body fat temperature assessment based on a calibration set that includes fat measured at different temperatures.

FIG. 12 illustrates using a multi-sample calibration measurement set to reduce analysis error due to constituent temperature variation.

FIG. 13 illustrates choosing the set of significant principal components in the calibration data based on a comparison of eigenvalues of a covariance matrix with eigenvalues obtained from measuring system noise.

FIG. 14 illustrates criteria for choosing the set of significant principal components in the calibration data based on minimizing measurement errors.

FIG. 15 is a graph of NMR determined hydration as contrasted with hydration determined by freeze-drying analysis.

### DETAILED DESCRIPTION

1. NMR Measurement Apparatus

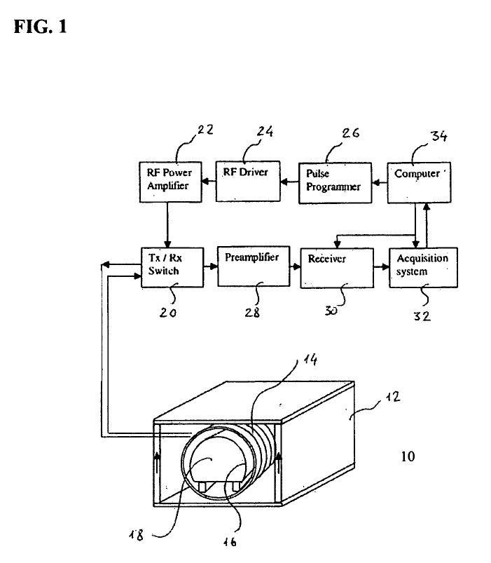

One embodiment of a nuclear magnetic resonance (“NMR”) apparatus according to the invention is shown generally in FIG. 1 at 10. The apparatus 10 includes a magnet 12 disposed around or on opposed sides of a sample chamber 18. The magnet 12 may be a permanent magnet, or an electromagnet, and is configured to induce a substantially homogeneous static magnetic field within the sample chamber 18. The volume of the sample chamber 18 may be defined by an enclosure such as a polycarbonate tube or box, shown generally at 16 in FIG. 1. The purpose of defining the chamber volume using the enclosure 16 is to precisely set the geometric boundaries of the volume in space within which a body being analyzed may be positioned and may even be moving during measurements, without substantially affecting accuracy of NMR measurements performed according to the invention. The enclosure 16 may be made from any substantially electrically non-conductive and non magnetic material known in the art.

A radio frequency (“RF”) antenna 14 is disposed about the enclosure 16, typically on the exterior surface of the enclosure 16. In the present embodiment, the antenna 14 comprises a coil wound so that its turns lie in planes approximately perpendicular to the longitudinal axis of the chamber 18. When pulses of RF electrical power are passed through the antenna 14, an RF magnetic field is induced within the chamber 18. Although described above in terms of coils, the antenna 14 can be configured in any other way as long as the RF magnetic field induced by the antenna 14 is substantially perpendicular to the static magnetic field induced by the magnet 12 within the volume defined by the chamber 18.

In the present embodiment, the antenna 14 performs both RF transmit and RF receive functions, and can therefore be coupled to a T/R matching circuit and switch 20. The switch 20 is under control of a computer 34 or similar programmable controller configured to operate the switch 20 such that the antenna 14 is selectively coupled to an RF power amplifier 22 during RF pulse transmission intervals, or to a receiver preamplifier 28 during NMR signal detection (receive) intervals. The input of the RF power amplifier 22 is coupled to an RF driver 24, the input of which is itself coupled to a pulse programmer 26. The pulse programmer 26 may be a separate element under control of the computer 34, or may be a function performed by the computer 34 itself.

The receiver preamplifier 28 is coupled to an RF receiver 30, which is itself coupled to an analog to digital converter (“A/D”) 32. The output of the A/D 32 is coupled to the computer 34 for analysis of voltages detected by the antenna 14 resulting from NMR phenomena in an object (not shown in FIG. 1), i.e. a human body, disposed within in the enclosure 16.

The pulse programmer 26 is configured to operate the RF driver 24 to cause generation of a succession of selected length and selected frequency RF pulses through the antenna 14, such that NMR phenomena are induced in the object (not shown). As is well known in the art, the frequency, amplitude and duration of the RF pulses are related to the amplitude of the static magnetic field within the chamber 18, and to the Larmor frequency of nuclei which are excited within the object (not shown) for NMR relaxometry analysis. For analysis of human bodies in particular, the nuclei are typically protons (¹H).

In the present embodiment, the RF pulse amplitude and duration can be selected to provide first approximately 90 degree (transverse) reorientation of magnetic spin axes of the protons in the object (not shown) and then a succession of 180 degree (inverse or refocusing) magnetic spin reorientations. Each refocusing RF pulse is typically followed by a time interval during which the antenna 14 is coupled to the receiver pre amplifier 28 for detecting NMR phenomena originating from within the object (not shown). Such sequences of transverse reorientation, inverse reorientation and NMR signal detection are well known in the art for determining transverse relaxation time (T₂) and longitudinal relaxation time (T₁) of materials being analyzed.

Certain aspects of the foregoing description of NMR apparatus and methods are well known in the art. In the invention, however, it has been determined that if certain requirements are observed for the amount of spatial variation of the static and RF magnetic fields within the sample chamber 18, and certain requirements for the excitation spectrum of the RF magnetic field are met, high precision can be obtained without the need to build a measuring apparatus of excessive size and cost. At the same time, apparatus and methods according to the invention which meet such requirements of static magnetic field distribution and RF field spatial distribution and spectral content are fully able to make precise measurements of whole body composition of, for example, a live, conscious animal or human subject, even if the body being analyzed moves within the enclosure 16.

Apparatus and methods according to the invention make practical for the first time analysis of living, conscious animals, including humans, for whole body composition without the need for large, expensive NMR spectroscopy or MRI (imaging) systems. In the invention, NMR measurements on human subjects the field of view cover the whole body and no displacement or movement of the body during measurements is required.

In order to explain the function of the invention, first, factors which affect the accuracy of NMR measurements will be explained. An expression for the NMR signal amplitude S(r₀, t) induced in an NMR receiver antenna (e.g., antenna 14 in FIG. 1) as a result of inducing NMR phenomena in an object or body being analyzed is as follows:

$$S ( {r}_{0} , t ) = {ω}_{0} \cdot {\int }_{{V}_{b}} {\sum }_{i} {m}_{i} ( {r}^{->} , t ) \cdot A ( {r}^{->} - {{r}^{->}}_{0} ) d V \qquad ( 1 )$$

where ω₀ is the NMR excitation frequency; A(\vec{r}−\vec{r}₀) is the NMR receiving antenna spatial sensitivity function and mᵢ(\vec{r},t) is the nuclear magnetization of i-th body material (substance, such as fat, lean, or water) component as a function of time and position of the elementary volume dV inside the chamber 18. \vec{r}₀ represents position of the center of the object or body. The nuclear magnetization can be further presented in the form:

mᵢ(r,t)=m₀ᵢ(\vec{r},t)·k(\vec{r}) (2)

where m₀ᵢ(\vec{r},t) represents magnetization, as function of position and time, of the nuclei in the i-th body component under idealized conditions of perfectly homogeneous excitation. k(\vec{r}) is a coefficient representing inhomogeneity of nuclear magnetic excitation conditions at every point in space within the chamber 18. The coefficient k(\vec{r}) depends on the spatial distribution of the static magnetic field and the RF magnetic field, the frequency spectrum of the RF magnetic field, the frequency spectrum of nuclear magnetic spins in the object being analyzed, and the RF receiver system frequency response (bandwidth). k(\vec{r})=const represents the condition where the nuclear magnetic excitation conditions are uniform over the entire chamber 18. This means that if the chamber 18 is filled with a homogeneous material, the magnetization of the material is spatially uniform.

The quantity of interest in body composition measurements is:

$${\sum }_{i} {\int }_{{V}_{b}} {m}_{0 i} ( {r}^{->} , t ) d V = {\sum }_{i} {M}_{i} ( t ) , \qquad ( 3 )$$

where V_{b} represents the body volume.

In the case of homogeneous magnetization m₀ᵢ(\vec{r},t)=const for (\vec{r} ∈ V_{b}), then equations (1) and (2) allow for describing the NMR signal in the form:

$$S ( {{r}^{->}}_{0} , t ) = [ {V}_{b} \cdot {\sum }_{i} {m}_{0 i} ( {r}^{->} , t ) ] \cdot ( 1 / {V}_{b} ) \cdot {\int }_{{V}_{b}} k ( {r}^{->} - {{r}^{->}}_{0} ) \cdot A ( {r}^{->} - {{r}^{->}}_{0} ) d V ∝ {\sum }_{i} {M}_{i} ( t ) \qquad ( 4 )$$

Equation (4) shows that the NMR signal amplitude from a homogeneous and homogeneously magnetized object or body is directly proportional to the quantity of the particular material of interest. Any movement of the object or body will not affect the total signal amplitude and will not affect the ratio between signal components.

Homogeneous composition is clearly not the case for inhomogeneous objects such as a living organism with naturally distributed fat and lean tissue (m₀ᵢ(r,t)≠const). The conditions for the NMR signal to represent true body composition in this case are k(\vec{r}−\vec{r}₀)=const and A(\vec{r}−\vec{r}₀)=const such that:

$$S ( t ) = const {\sum }_{i} {\int }_{{V}_{b}} {m}_{0 i} ( {r}^{->} , t ) d V ∝ {\sum }_{i} {M}_{i} ( t ) \qquad ( 5 )$$

Therefore, embodiments of a method and apparatus according to the invention minimize spatial variation of the coefficient k and minimize spatial variation of the antenna sensitivity function A with respect to any particular size of sample chamber. It will be readily appreciated by those skilled in the art that similar results, as they pertain to accuracy and speed of measurement, could be obtained for body composition analysis by using NMR measurement systems and techniques known in the art. For example, well known NMR laboratory composition analysis systems have, in the centermost portions of their sample chambers, antenna sensitivity distribution and static magnetic field homogeneity such that accurate composition analysis can be made on inhomogeneous and/or moving objects over a very small volume. In fact, such systems known in the art have been used successfully to perform body composition analysis of very small laboratory mice. However, the structures of such known in the art apparatus would be impractical to increase in size in order to perform similar whole body composition analysis on much larger animals, for example rats, dogs or even humans. Embodiments of methods and apparatus according to the invention provide accurate whole body composition of much larger animals but maintain a practical size and weight of the overall apparatus.

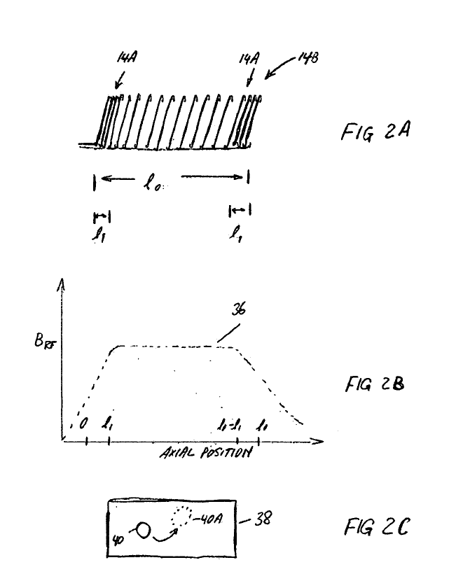

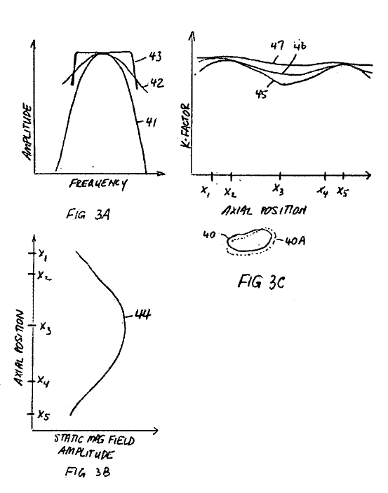

FIG. 2A shows an example of an antenna that generates an RF magnetic field having inhomogeneity of less than about 2% over the entire volume of the chamber (18 in FIG. 1). The antenna coil 14B has a total length along its longitudinal axis represented by l₀. Over the central portion of the antenna coil 14B, coil windings have a first “turn density” (number of turns per unit length along the axis). At each longitudinal end of the antenna 14B is a “booster coil”, shown at 14A, each of which has a selected length along the axis represented by l₁, and a turn density of about twice that of the central portion. Preferably, the axial length, l₁, of each of the booster coils 14A is about 0.125 the total axial length l₀ of the antenna 14B. It will be readily appreciated by those skilled in the art that reduced RF field inhomogeneity could be obtained by increasing the axial length of the antenna with respect to the axial length of the sample chamber. Advantageously, an antenna configured as shown in FIG. 2A and as described above provides reduced RF field inhomogeneity while maximizing the effective sample chamber length with respect to the total antenna length along respective longitudinal axes. In another embodiment of the antenna, the coil 14B may be center tapped. The center tap may be connected to one terminal of the switch (20 in FIG. 1). The ends of the coil, which may be the longitudinal ends of the booster coils 14A, may both be connected to the other terminal of the switch (20 in FIG. 1).

The RF magnetic field distribution along the longitudinal axis of the antenna coil 14B is presented in FIG. 2B. Due to the reciprocity principle, the spatial distribution of the RF magnetic field represented in FIG. 2B should be substantially the same as the spatial distribution of the antenna sensitivity function, when the same antenna is used for both RF magnetic field generation and NMR signal reception. FIG. 2C shows that an inhomogeneity 40 disposed within the axial limits 38 defined by the chamber (18 in FIG. 1) can move, such as shown at 40A in FIG. 2C, and still induce a substantially equal amplitude incremental NMR signal component in the antenna (14 in FIG. 2A). The inhomogeneity 40 may be a portion of an entire body of an animal or human subject able to move within the enclosure (16 in FIG. 1), or may represent the entire animal subject disposed in an enclosure larger than the animal itself.

The foregoing description with respect to FIGS. 2A, 2B and 2C explains an antenna structure intended to minimize spatial variation of the antenna sensitivity function A (from equation (5) above). FIGS. 3A, 3B and 3C will be discussed below with respect to aspects of the invention related to minimizing spatial variation in the coefficient k (from equation (5) above).

Referring first to FIG. 3A, which is a graph of amplitudes of various frequency components in RF pulses used to induce the RF magnetic field, using conventional RF pulses, shown by curve 41, the frequency spectrum of the RF magnetic field induced by these pulses transforms into spatial variation of excitation conditions, or coefficient k(\vec{r}) when the static magnetic field is not uniform. Variation in static magnetic field amplitude with respect to axial position is shown at curve 44 in FIG. 3B. The excitation coefficient k(\vec{r}) with respect to axial position x, corresponding to the static magnetic field variation (44 in FIG. 3B) and conventional RF pulse bandwidth (41 in FIG. 3A) is shown at curve 45 in FIG. 3C. Referring back to FIG. 3A, if the length of the RF pulses is shortened, the bandwidth of RF energy in the pulses is increased, as shown curve at 42. As is well known in the art, the RF pulses can be increased in amplitude in order to maintain the same amount of reorientation (same angular displacement) of the nuclear magnetic spin axes if the pulse duration is shortened. Curve 46 in FIG. 3C shows reduced variation of the coefficient k(\vec{r}) with respect to position when the RF magnetic field has increased bandwidth. Another way to optimize the RF magnetic field spectrum is the use of composite RF pulses, a variety of which are explained in R. R. Ernst, et al., Principles of Nuclear Magnetic Resonance in One and Two Dimensions, Oxford University Press, 1987. Curve 43 in FIG. 3A shows an example bandwidth of composite RF pulses. Curve 47 in FIG. 3C shows very little variation in excitation with respect to position when composite pulses are used.

Yet another possibility to optimize the RF magnetic field in order to achieve better uniformity of nuclear magnetization over the volume of the chamber (18 in FIG. 1) is to use shorter duration RF pulses at unchanged amplitude with resulting lower magnetic spin axis rotation angle. The benefit of the wider frequency bandwidth and its effect on precision of measurements overweighs some of the disadvantage of a resulting loss in NMR signal amplitude because of reduced net transverse nuclear magnetization.

FIG. 3C illustrates the fact that in a linear approximation the coefficient k(\vec{r}) (or nuclear magnetization of a homogeneous object that fills all space within the object compartment) is simply the Fourier transform, or spectrum, of the RF pulse. The statement that uniform spectral density of RF pulse causes substantially uniform magnetization holds approximately true for the non-linear (typical) case as well. It can be observed in FIGS. 3A, 3B and 3C that better uniformity of the static magnetic field will also improve uniformity of the nuclear magnetization. It is to be noted, though, that merely attempting to improve the uniformity of the static magnetic field, without additional remedies, requires a dramatic increase in the size, weight and cost of the magnet, irrespective of the type of magnet being used. In the invention, therefore, optimizing the RF pulses properties and the spatial distribution of the antenna sensitivity can make it possible to use substantially smaller and less expensive magnets while still providing high accuracy and precision in NMR relaxometry measurements.

Spin rotation angle in the range between 90 and 180 degrees for the refocusing RF pulses is also beneficial from the point of view of saving power when a large object is under investigation. In the case of measurements performed on humans, the reduced power produces less heating and therefore is advantageous from a safety point of view.

In the description above it is assumed that the receiver channel (including antenna 14, switch 20, preamplifier 28 and receiver 30) has sufficient bandwidth in order to uniformly (uniform signal amplification with respect to frequency) receive signals from parts of the object (not shown) corresponding to different resonance frequencies of nuclear magnetic spins. Alternatively, the receiver channel can have a frequency response that compensates for non-uniform excitation due to inhomogeneity in the static magnetic field and the limited, non-uniform spectrum of the RF pulses.

An aspect of the present invention is a pulse sequence and a signal processing technique that further reduces the effects of the remnant small inhomogeneities of the nuclear magnetization (represented by the coefficient k(\vec{r})) in the volume of interest. As was explained above a typical measurement sequence comprises an excitation RF pulse producing 90 degree reorientation (excitation pulse) of magnetic spin axes from the equilibrium state and then a succession of 180 degrees (refocusing) magnetic spin reorientations. It is well known that a succession of a large number of RF pulses has a spectral content that includes essentially discrete frequency elements. The discrete frequency elements of such spectrum are separated by a frequency interval defined in the following expression:

$$Δ f = \frac{1}{T E} \qquad ( 6 )$$

where TE is the time interval between pulses in the RF pulse sequence.

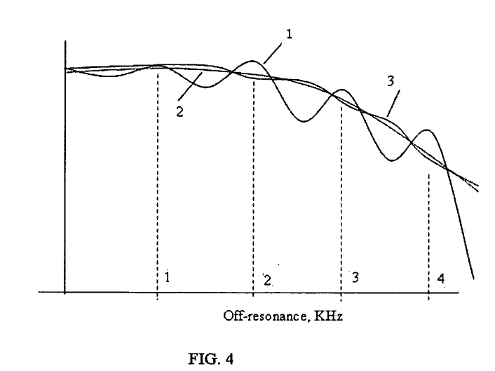

The discrete frequency elements cause ripples in the spectrum of the nuclear magnetization. The result of modeling of the nuclear magnetization is shown in FIG. 4, line 1 for TE=1 ms. The magnetization distribution has a main trend represented by line 2 and the ripples. Due to inhomogeneity in the static magnetic field, the frequency variation in the nuclear magnetization transforms into spatial variations in the amplitude distribution of the nuclear magnetization. Thus, one can expect stronger magnetization in the volumes where the static magnetic field corresponds to the spectral components of the RF pulse sequence and weaker magnetization in the regions where the static magnetic field corresponds to the frequency in the interval between the discrete frequency components. In order to reduce the amplitude of the ripples and their effect on the nuclear magnetization, the NMR measurement sequence is repeated at a series of N values of the carrier frequency forming an arithmetic sequence with difference

$$Δ {f}_{J} = \frac{Δ f}{N} = \frac{1}{N \cdot T E} \qquad ( 7 )$$

and the average of the N resulting NMR measurement sequences is taken

In the simplest case of N=2, shown as line 3 in FIG. 4, as a result of repeating the NMR measurement sequence using different RF carrier frequencies as explained above, the stronger and weaker magnetized regions substantially switch positions in space so that the average of the foregoing NMR measurement sequences gives a much smoother spectrum of the nuclear magnetization and correspondingly much smaller spatial inhomogeneity of the magnetization amplitude.

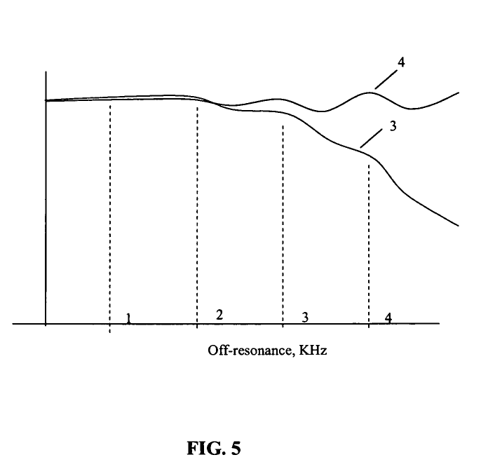

As explained above, effects of non-uniform excitation caused by the limited bandwidth of the RF pulses can be compensated by using a correction filter in the receiver channel. The result of using of the filter is illustrated by FIG. 5 at line 4, as contrasted with the response not using the correction filter, as shown by line 5.

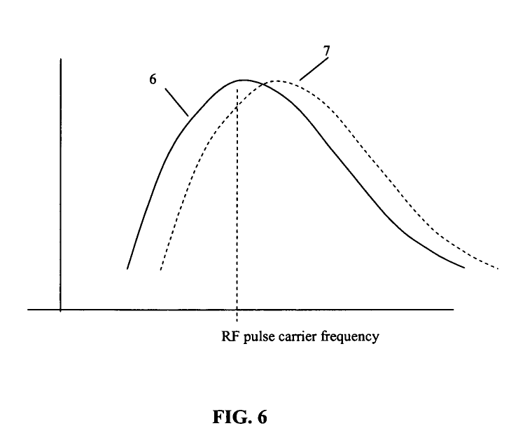

The distribution of value of the static magnetic field in a body positioned for measurement can slightly vary from measurement to measurement, in shape and in its characteristic value (which can be mean, median, mode or another statistical property elected to serve as characteristic). These variations are determined mainly by the following two independent sets of circumstances One is the shape and the positioning of different bodies as well as the positioning of the same body inside the magnet, and another is temporal variations in the static field induced by the magnet, such as those caused by ambient temperature drifts and by redistribution of stresses in the magnetic materials. Whatever the causes of the changes in the distribution of amplitude of the static magnetic field in a body, the resulting measurement errors can be minimized by bringing the RF pulse carrier frequency to a value such that the most represented in the body values of the Larmor frequency lie closest to the carrier frequency, as illustrated in FIG. 6. Curves 6 and 7 in FIG. 6 show examples of two instantaneous positions of the distributions of Larmor frequency of a body corresponding to the distributions of the static magnetic field. The desired position of the carrier frequency corresponding to the distribution at curve 6 is indicated by the vertical dashed line. To this end, the NMR signal acquired from the body is analyzed to find the best relative positioning for the RF carrier frequency. Then, either the carrier frequency is adjusted or, in the case of an electromagnet being used to induce the static magnetic field, the magnet current is adjusted to achieve the best relative positioning. One possible implementation of such an adjustment is to first make a measurement of spin echoes of several pulses and then perform a least square fit of the phase of the accumulated signal to a linear function of time. The slope of the linear function yields the required correction to the carrier frequency. Another possible implementation of such an adjustment is to first make a measurement of echoes of several pulses, find the maximum of a smoothly defined analog to the spectrum of the accumulated signal and take this maximum as an optimal relative position for the RF carrier frequency.

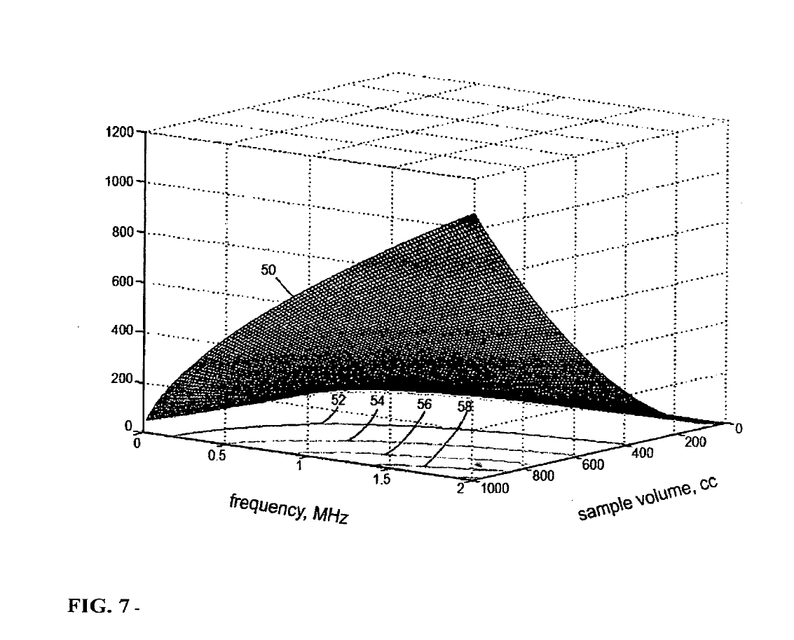

An important relationship exists between the size of the object or body to be analyzed (related to the sample chamber volume), and the choice of NMR operating frequency (the frequency of the RF pulses applied to the antenna). As is well known in the art, the NMR frequency is proportional to the static magnetic field intensity and the gyromagnetic ratio of the nuclei being analyzed. The relationship between operating frequency and size of the body being analyzed can be used in various embodiments to select a minimum strength static magnetic field, and corresponding NMR frequency, which will provide measurements having acceptable accuracy and precision. FIG. 7 shows a three-dimensional graph, at surface 50, of the signal-to-noise ratio (SNR) with respect to the sample (object or body) volume and the NMR operating frequency. For a particular value of SNR, as required to perform selected duration and yet accurate NMR measurements, there is a relationship between the minimum NMR frequency that facilitates obtaining the required accuracy with respect to the volume of the sample inside the chamber (18 in FIG. 1) wherein the object is placed. The relationship for selected values of SNR is shown by curves 52, 54, 56 and 58 in FIG. 7. Each curve represents the relationship for a given (predetermined) SNR level. Curve 54, for example, represents the minimum NMR frequency as it relates to the selected sample volume for SNR of 100. As will be appreciated by those skilled in the art, longer duration NMR measurements sequences may be used with lower SNR. The value of SNR selected will thus be related to the speed with which NMR analysis needs to be performed on any particular type of object. Irrespective of the SNR selected, the relationship between sample volume and minimum NMR frequency can be used to minimize, for any selected chamber volume, the strength of the magnet used to induce the static magnetic field. Designing NMR system with minimum NMR frequency thus gives benefits of reducing the size, weight and cost of the magnet assembly for any particular sample volume. The sample is expected to be disposed entirely within the chamber.

All of the foregoing attributes of an apparatus according to the invention are used to maximize the volume of objects being analyzed with respect to the physical dimensions (and associated cost) of the NMR measurement apparatus itself. This is in contrast to apparatus known in the art which must be scaled up, or increased in size (and associated cost) in order to make NMR measurements of a selected accuracy on larger and larger objects.

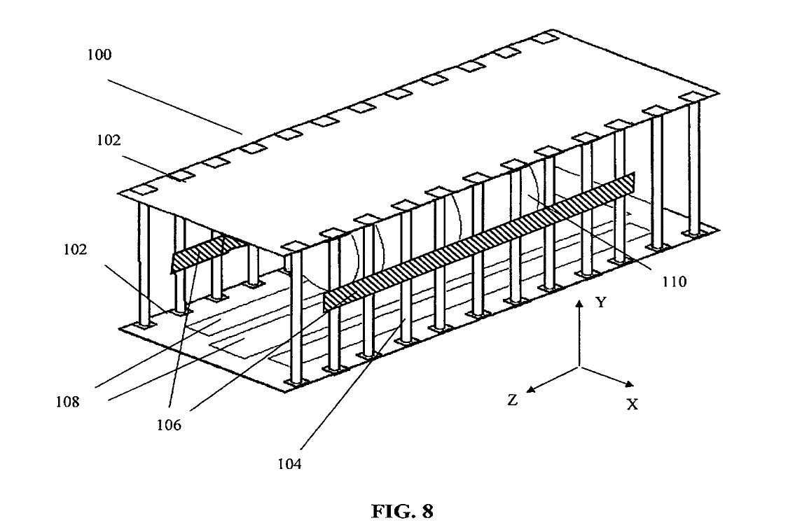

FIG. 8 shows the details of one possible implementation of an electromagnet assembly according to the present invention that is used to induce the static magnetic field within the chamber (18 in FIG. 1). The electromagnet assembly 100 comprises pole pieces 102 substantially in the form of flat plates, magnetizing coils 104 with iron cores wound so that their magnetic field is substantially along the Y-axis shown in FIG. 8, side shims 106 preferably made of soft magnetic steel, and top/bottom shims 108 also preferably made of soft magnetic steel. The static magnetic field is generated by passing direct current (DC) through the magnetizing coils 104 from a stable current source. The magnetizing coil 104 arrangement and the sets of side 106 and top/bottom shims 108 allow for simple, inexpensive, and substantially orthogonal shimming of the magnet 100, that is to adjust the magnetic field homogeneity consecutively in X, Y and Z axes directions. The adjustments substantially do not affect each other. The width (dimension in Y-axis direction) of the side shims 106 is chosen to minimize inhomogeneity along the X-axis direction. The thickness of the top/bottom shims 108 on the pole pieces 102 is adjusted to minimize inhomogeneity along the Y-axis direction. When substantial homogeneity in the XY center plane is achieved, homogeneity along the Z-axis direction can be adjusted by selecting current magnitude in each of the magnetizing coils 104. Typically, the current amplitude is higher in the longitudinal end most magnetizing coils and is lower in the centermost coils. In order to set different current magnitude in each of the coils 104, individual coils 104 can be driven from a separate DC power supply. The magnetizing coils 104 can be also connected in electrical series. In this case separate current adjustment in the coils can be achieved by using selected and/or variable resistors connected in parallel to each coil. The capacity to set a different current in each of the plurality of the magnetizing coils 104 provides the magnet assembly 100 with very fine field adjustment capability in order to correct for any inhomogeneity in the static magnetic field caused by, for example, magnetic material inhomogeneity or imprecision in mechanical assembling of the magnet assembly 100. As previously explained with reference to FIG. 6, by using one or more electromagnets in the form of coils or other form, it is possible to adjust the overall amplitude of the static magnetic field to cause the NMR signal spectrum to substantially match the RF pulse carrier center frequency.

In some embodiments, the current through all of the coils 104 may be adjusted to provide, for different NMR experiments, more than one principal static magnetic field magnitude. A first NMR experiment may be performed as explained further below at a first static magnetic field magnitude. The current through all the coils 104 may then be adjusted to change the magnitude of the static magnetic field. As will be appreciated by those skilled in the art, when the static magnetic field magnitude is changed, the RF excitation frequency will need to be changed correspondingly in order to induce NMR phenomena in the body being analyzed. A second NMR experiment may then be conducted on the body disposed in the apparatus as explained further below. By conducting the NMR experiments at two distinct frequencies, it is possible to determine two distinctly different relaxation processes for the substances being analyzed.

Each of the coils 104 may be further split into two parts (not shown in FIG. 8). One part of each coil 104 will generate a homogeneous magnetic field, and the other part will generate a gradient (in the axial direction). The gradient can have a variable strength. The foregoing feature enables body composition analysis within an axial slice at a selected position along the longitudinal axis of the apparatus. At a given RF frequency, the gradient strength will determine the slice thickness, and the static magnetic field strength will determine the axial position of the center of the slice.

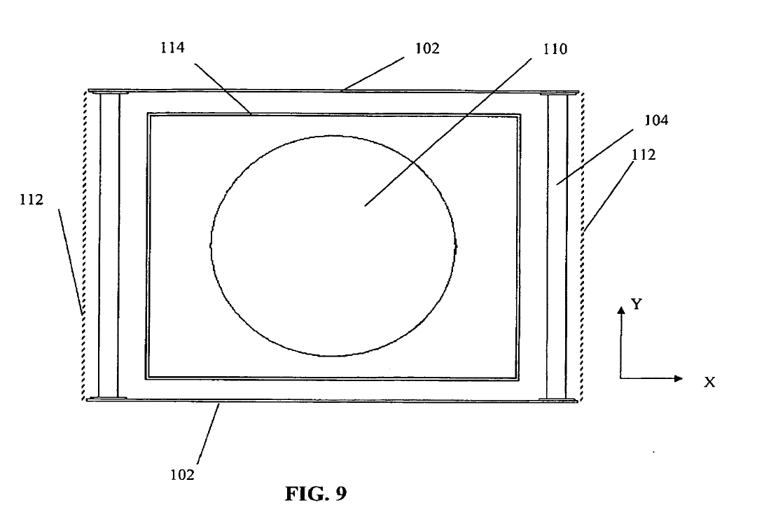

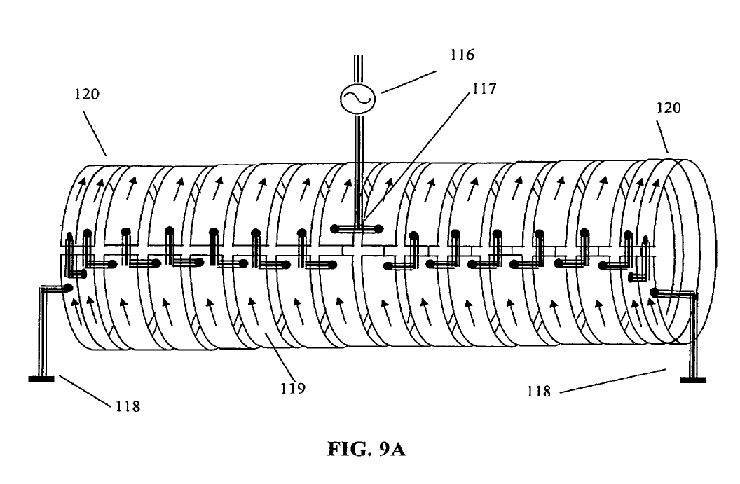

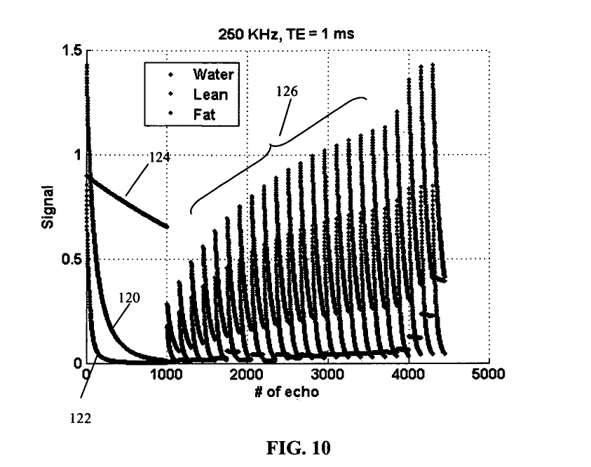

FIG. 9 is a magnet/antenna assembly view from the front showing the RF shielding parts. The RF shield can be made from two electrically conductive, preferably aluminum, side pieces 112, and the previously described magnetic pole pieces 102. The RF shield is completed by two front/back pieces (not shown in FIG. 9). The role of the front/back pieces is twofold, first to achieve better shielding of the sensitive region 110 (field of view) from any external RF energy sources, and 2) to comply with United States Federal Communication Commission regulations (and corresponding regulations outside the United States) on RF radiation from the antenna. In many cases the shielding of the sensitive region is sufficient by just using the pole pieces 102 and side pieces 112. The RF dipole causing undesirable radiation far from the antenna can be effectively eliminated using an active RF spoiler 114 that leaves free access to the sensitive region 110. The spoiler 114 can be implemented as a RF current loop connected to the same transmitter as the main antenna. The current in the spoiler loop 114 is adjusted so as to substantially reduce the total RF dipole emanating from the antenna.

FIG. 9A shows an antenna arrangement that can accommodate large objects like an obese human. RF magnetic field homogeneity requires that the antenna current density be evenly distributed over the antenna length. It would be clear for those skilled in the art that single serial windings of such a large antenna would have too high an inductance and consequently require too high a voltage on the antenna terminals to be practical. Such high voltage would complicate the antenna driver circuitry and would complicate compliance with safety regulations. In order to reduce the antenna inductance, the antenna can be made from two parallel sections with the windings made as shown in FIG. 9A. The voltage source 116 is connected between a center tap 117 and ground terminals 118 of the two antenna sections. The current in both sections flows in the same direction, as indicated by reference numeral 119. Shown at 120 in FIG. 9A are end turns having twice higher current density as compared to the main part of the antenna windings. The end turns improve the homogeneity of the RF magnetic field in transmit mode and the antenna sensitivity in receive mode.

In a method according to the invention, a live, conscious animal (human subject) is placed in the enclosure (16 in FIG. 1). The magnet (12 in FIG. 1) induces a static magnetic field in the animal. RF pulses according to a programmed sequence are passed through the antenna (14 in FIG. 1), between which pulses, NMR signals are detected by the antenna. A record is made of the NMR signals thus detected, and from the detected NMR signals, composition of various components of the animal body are analyzed. In one embodiment, the RF pulses passed through the antenna form the well known Carr-Purcell-Meiboom-Gill (CPMG) sequence. Body component composition may be determined from the total spin echo amplitude train detected. Methods for determining contributions of components of various relaxation characteristics in a spin echo amplitude decay spectrum are well known in the art for both T₁ and T₂ relaxation measurement techniques.

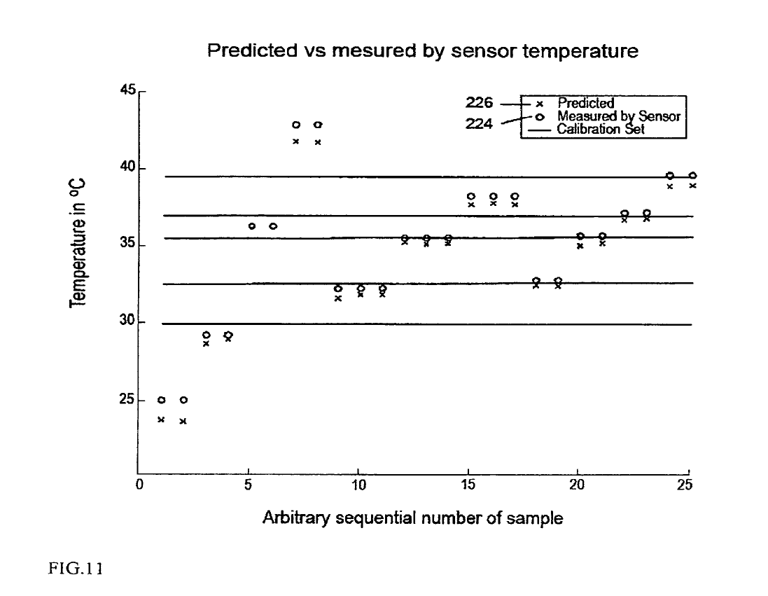

One implementation of a method according to the invention is an NMR measurement technique that enhances the contrast between types of human body tissues to be differentiated. FIG. 10 represents CPMG spin echo sequences comprising a plurality of CPMG sub-trains each separated by the following recovery delay times (expressed in milliseconds): 20, 30, 40, 50, 60, 70, 80, 90, 100, 110, 120, 130, 140, 150, 160, 170, 180, 190, 200, 250, 500, and 1500. The points making the curves in FIG. 9 are the amplitudes of the spin echoes. The decay rate in each sub-train represents the transversal (T₂) relaxation, while dependence of amplitudes (126) of the relaxation curves on the recovery time reflects the longitudinal relaxation (T₁). FIG. 10 corresponds to NMR relaxation measurements made at an operating frequency of 250 KHz. The results represent constituents of a human body: fat tissue 120; lean tissue 122; and free water 124. Sufficiently high tissue signal contrast with respect to noise on the T₂ relaxation curves at 250 KHz suggests that fat tissue, lean tissue and free water can be effectively differentiated. It is important to note that the lower the NMR operating frequency, the less expensive the implementation of NMR measurement system for the whole body composition measurement, primarily because the size of the magnet for the static magnetic field is reduced. It is practical to use an electromagnet that does not require cooling because of the relatively low static magnetic field amplitudes. A possible additional benefit of using relatively low static magnetic field amplitude and corresponding low RF excitation frequency is reduced environmental noise caused by the apparatus. A preferred frequency for NMR measurements according to the invention is at most about 500 KHz, and more preferably in a range of 250-350 KHz.

In a method according to the invention, the RF frequency used is relatively low as contrasted with typical frequencies used in medical imaging. One of the reasons for using a relatively low frequency is the requirement to have as little attenuation of the NMR signal as possible due to eddy current losses. There is a substantial difference between the importance of signal attenuation as it relates to ordinary imaging and the importance of signal attenuation as it relates to quantitative analysis as in the present invention. In the case of ordinary imaging, mere signal contrast between adjacent voxels is sufficient for creating a usable image. However, for quantitative analysis with an expected precision of at least 1%, signal magnitudes should not be distorted by more than about 1%. It is well known that the RF magnetic field and will attenuate when passing through animal or human body tissue because the tissue is electrically conductive. The degree of attenuation depends on the electromagnetic properties of the tissue and the thickness of the tissue. Both properties are highly variable within a typical human subject, and are also highly variable between individual human subjects. Typically, the RF skin depth in human soft tissues is about 0.6 m at 1 MHz. The skin depth, as known in the art, is inversely proportional to square root of the frequency. It has been determined experimentally that for a typical human body the RF attenuation is less than 0.1% at a frequency of 0.3 MHz. In a method according to the invention, a time varying electromagnetic field used to investigate a property of a human subject is selected such that electromagnetic field attenuation is at most equal to a selected precision with which the property is to be measured, and more preferably is at most about one-tenth the selected precision. In the present invention, the selected precision as explained above may be about 1 percent. A field attenuation at the preferred frequency is at most about 0.1 percent.

The apparatus shown in FIG. 1 may have a sufficiently large chamber and enclosure so as to accommodate extremely obese patients or pregnant female patients for evaluation, for example, as much as 500 pounds body weight. The enclosure may be configured on rollers or similar movable device such that a human patient may be introduced into the machine by rolling or sliding the enclosure into the chamber. Such rolling or sliding, linearly movable enclosure also can make it possible the use of the apparatus to examine materials using a single direction of motion conveyor, for such purposes as screening for explosives or other contraband.

Other possible advantages of an apparatus made as explained above are that no RF shielding room is required, because of the relatively low RF excitation frequency used, and no magnetic shielding is required, because a static magnetic field of 5 Gauss or less is experienced within the limits of a typical room used to house the apparatus and perform experiments on human patients and laboratory test animals. The low RF magnetic field noise induced externally to the apparatus and relatively low static magnetic field amplitude make possible location of the electronic circuitry portion of the apparatus (shown in FIG. 1) proximate the magnet and RF antenna, for example, under a patient bed which may be slidable longitudinally into the chamber.

2. Analysis of Body Composition Using NMR Measurements

In one embodiment of a method according to the invention, and using an apparatus as explained above with respect to FIG. 1, a live, conscious animal (depending on the size of the apparatus, this may be a human infant or adult) is placed in the sample chamber (18 in FIG. 1). The magnet (12 in FIG. 1) induces a static magnetic field in the animal. RF pulses according to a programmed sequence are passed through the antenna (14 in FIG. 1), between which pulses, NMR signals are detected by the antenna (14 in FIG. 1). A record is made of the NMR signals thus detected, and from the detected NMR signals, composition of the animal body is analyzed. In one embodiment, the RF pulses passed through the antenna have duration, amplitude, phase, and spacing between successive RF pulses to form the well known Carr-Purcell-Meiboom-Gill (CPMG) sequence. The signals detected using a pulse sequence constitute nuclear magnetic spin echoes. Body composition may then be determined from the properties of the spin echoes in particular arrangements of sequences as will be explained further below.

NMR data suitable for body composition analysis according to the invention are obtained from a suitable arrangement of measurement sequences including a plurality of CPMG sequences. The first CPMG sequence is relatively long, and is followed by a purality of relatively shorter length CPMG sequences. Each CPMG sequence, both long and short, includes an initial transverse magnetic polarization pulse followed by a selected number of inverting or refocusing pulses. “Long” and “short” as used herein with respect to the CPMG sequences in the measurement arrangement relate to the number of refocusing pulses used in the various CPMG sequences. One suitable arrangement of measurement sequences is represented in FIG. 10 described above.

Each CPMG sequence generates NMR spin echo data that can be used to determine transverse nuclear magnetic relaxation properties, such as the T₂ relaxation time. Recovery times D₁, D₂, . . . Dᵢ, between successive CPMG sequences are selected to be comparable to the longitudinal magnetic spin recovery time of the constituents of the body, so that the relative amplitudes of the spin echoes detected in each of the sequences can be used to determine longitudinal nuclear magnetic relaxation properties. Thus, the example CPMG sequence arrangement described herein provides spin echo data that can be used to determine both transverse and longitudinal relaxation nuclear magnetic properties of the body (or body part) being analyzed.

It has been determined experimentally that NMR measurements having identifiable transverse and longitudinal relaxation components can improve the analysis of constituent composition of a body or body part as compared to using either transverse or longitudinal relaxation components alone. The overall transverse and longitudinal relaxation properties of the body (or body part) being analyzed, as reflected in the spin echoes measured as explained above, will reflect the respective masses of, and the relaxation properties of, certain constituents of the body being analyzed. In methods according to the invention, the mass (or fractional amount) of each of a selected number of constituents can be determined from the spin echoes. The following is an explanation of how this is performed according to the invention.

Methods according to the various aspects of the invention determine an amount (mass or fractional amount) of one or more selected constituents (e.g. fat, lean tissue and free water) in a body or body part subject to NMR measurements by calculating a predetermined function with respect to the NMR measurements. The function for each constituent is determined from a standard which represents each constituent. A generalized standard for a body constituent in the present invention is a set of substances that represents substantially all possible compositional and temperature variations of the represented body constituent (e.g., fat, lean tissue or free fluids) in a real object (live animal or human). An example of a set of substances that defines a standard for body fat can include vegetable oils such as olive oil, canola oil and sunflower oil in various proportions and at different temperatures. The temperatures are typically in the range of about 30-40° C. A lean tissue standard may include chicken breast muscle tissue at different temperatures, as well as synthetic porous media. One example of such synthetic porous media includes substances (gels) sold under the trade name SEPHADEX G-15 or SEPHADEX G-25, by Pfizer, Inc., New York, N.Y. These substances model water in biological tissues. Alternatively, a standard for lean tissue may be determined using dual energy x-ray analysis of human lean tissue samples. For human body composition analysis, canola oil is preferred as a standard to represent fat tissue response.

It has been determined through laboratory experiments that for a given NMR measurement set (presented in the detailed description of the embodiments of the present invention) the measured NMR signals (measurement vectors) obtained on the standards corresponding to different constituents substantially do not overlap. This is a prerequisite for a successful differentiation between the body constituents. Ways to implement the differentiation are presented in the description which follows. An important aspect of methods according to the present invention is that a total amount of fat, irrespective of the type and/or distribution of fat in a body part, can be calculated as a predetermined function of the NMR measurements of the body or body part. The predetermined function represents calibration measurements made on a set of test substances, such as the aforementioned canola, olive and/or sunflower oils made at various temperatures. Thus, methods according to the invention make it possible to determine the total fat amount or mass within the body part without the need to compositionally analyze the various fat types within the body part or within other body parts to be analyzed. As a result, methods according to the invention enable rapid, in-vivo fat mass or content determination without the need for difficult and expensive compositional analysis.

The spin echo data from the NMR measurements made as explained above are used to construct a “measurement vector” whose components are calculated from the spin echoes. In one embodiment, each spin echo contributes to a single component of the measurement vector. For example, the component can be a convolution of the echo and a kernel. The kernel can be selected to represent a specific purpose, such as yielding an overall amplitude of the echo by averaging several measured values in the middle of an interval. In another embodiment, the kernel can play the role of a frequency filter acting to reduce the effects of residual small inhomogeneities of the static magnetic field in the volume of the body.

In one embodiment, the predetermined function is linear and its calculation is calculation of a scalar product of a measurement vector and a regression vector, namely, given a measurement vector V, the masses of a predetermined set of body constituents are obtained as follows. Based on pre-arranged calibration measurements, as will be further explained below, each constituent A has associated with it a regression vector R_{A}, of the same dimension as the measurement vector V. The mass of constituent A in the body or body part being analyzed is proportional to, or, in a simple version can be assumed to be equal to the scalar product V·R_{A}.

The set of regression vectors {R_{A}} for a set of constituents {A} is determined from a set of calibration measurement vectors. The calibration measurement vectors are obtained in an “a priori” calibration measurement procedure, wherein each regression vector R_{A} depends on the selection of constituents in {A}. R_{A} cannot be determined without the whole set of constituents {A} being defined first.

In some embodiments, the regression vectors {R_{A}} are obtained from some variant of least squares (LS) fitting of calibration vectors. The dimension of a regression vector is usually larger than the number of calibration vectors, and therefore the LS fitting must be preceded by a dimension reduction procedure. In one embodiment, which will be further explained later in this description, the dimension reduction procedure takes the form of restricting the regression vector to a subspace formed by the calibration measurement vectors. In another embodiment, which will be further explained later in this description, the regression vector is further restricted to a sub-subspace of the calibration measurement vector subspace by means of a principal component analysis (PCA).

In some embodiments, calibration vectors are smoothed in the following sense. The plurality of components of a calibration measurement vector in which a single component corresponds to one CPMG spin echo is regarded as a “regression function” of the consecutive number of the echo. This function is approximated by a piece-wise smooth function, such as, in one example, a sum of exponents with non-negative coefficients.

In one embodiment, the calibration set of measurement vectors comprises NMR spin echo measurements, made using the long and short duration CPMG sequences as explained above, corresponding to each of three selected major constituents of the body. The three selected body constituents in this example are fat tissue, lean tissue, and free water. The calibration measurement vectors may be averaged over a few separate sets of NMR calibration measurements made on each calibration sample to reduce the effects of random additive.

It has been determined experimentally that the NMR spin echo amplitude response of real constituents of the bodies of animals, such as mice and rats, as well as humans, can be adequately characterized with respect to quantities or fractional amounts of fat tissue, lean tissue and free water by making calibration measurement sets using canola oil to represent the fat tissue, using chicken breast muscle tissue to represent the lean tissue, and by using 0.9 percent sodium chloride (saline) solution to represent the body fluids consisting essentially of free water, such as urine. This is a particularly important finding with respect to characterization of fat tissue and lean tissue because of the compositional variations of such tissues within a living body.

In one embodiment, which can be designated “single-sample”, the spin echo amplitudes are used to create three calibration measurement vectors V_{fat}, Vₗₑₐₙ and Vₛₐₗᵢₙₑ, for fat tissue, lean tissue and free body fluids, respectively. In this embodiment, the following expressions are used to determine the regression vectors based on V_{fat}, Vₗₑₐₙ and Vₛₐₗᵢₙₑ that were normalized to 1 gram of mass, and averaged over several samples of each substance:

R_{fat}=Vₗₑₐₙ×Vₛₐₗᵢₙₑ[V_{fat}·(Vₗₑₐₙ×Vₛₐₗᵢₙₑ)]⁻¹, (8)

Rₗₑₐₙ=Vₛₐₗᵢₙₑ×V_{fat}[V_{fat}·(Vₗₑₐₙ×Vₛₐₗᵢₙₑ)]⁻¹, (9)

Rₛₐₗᵢₙₑ=V_{fat}×Vₗₑₐₙ[V_{fat}·(Vₗₑₐₙ×Vₛₐₗᵢₙₑ)]⁻¹, (10)

where the cross-product is defined as a usual three-dimensional cross product in three-dimensional linear sub-space, extended over the three calibration measurement vectors, Vₛₐₗᵢₙₑ, Vₗₑₐₙ, and V_{fat}.

In other embodiments, which are designated “multi-sample”, in order to improve the accuracy of the results of the analysis, the set of calibration measurements used to generate the regression vectors for any one or more of the constituents can include making calibration measurements on more than one sample of a particular constituent. For example, measurements made on the same physical sample of a constituent may be made at different temperatures. Another variation includes making calibration measurements on different samples of the same substance representing the same body constituent, for example, different types of oil, or different samples of animal lean muscle tissue. The use of multi-sample calibration measurements sets reduces composition analysis error due to factors such as natural variations in the chemical composition of a particular body constituent, or variation in the body temperature, each of which may result in slightly different NMR relaxation properties for the same constituent.

In “multi-sample” embodiments where the regression vectors are calculated from measurements made on multiple samples and/or measurements made at multiple temperatures, there will be several calibration measurement vectors for each basic substance (constituent). The respective sets of vectors are denoted as Vₛ={V_{saline, i}; i=1, . . . , Nₛ}, V₁={V_{lean, i}; i=1, . . . , N₁}, and V_{f}={V_{fat, i}; i=1, . . . , N_{f}}, where Nₛ represents the total number of free water calibration measurement vectors, N₁ represents the total number of lean tissue calibration measurement vectors, and N_{f} represents the total number of fat tissue calibration measurement vectors. The complete set of calibration measurement vectors Vₐₗₗ={Vₛ, V₁, V_{f}) contains the total of Nₐₗₗ=Nₛ+N₁+N_{f} calibration measurement vectors.

The canola oil and saline solution samples, used to produce calibration vectors for fat and free water, respectively, can be well standardized with respect to chemical composition. Therefore, differences in NMR response for various samples of canola oil and saline solution will more closely reflect differences such as temperatures rather than differences in chemical composition. On the other hand, at the present time, a method for creating a stable (compositionally uniform) laboratory standard for the chicken breast muscle tissue to represent lean body tissue (or other substance used to represent lean body tissue) is not yet established. As a result, different samples of chicken breast tissue may noticeably differ in chemical composition. The differences in the NMR signal response caused by differences in composition and by different constituent temperatures are of comparable magnitudes for various samples of chicken breast tissue. In one example, to reduce errors in body composition analysis, more than 100 different samples of chicken breast muscle tissue were used to generate the set of calibration measurement vectors for lean tissue, V₁.

In one “multi-sample” embodiment, the principal component analysis (PCA) is applied to the set of calibration measurement vectors, Vₐₗₗ, in the following form. An arbitrary orthonormal basis B={Bⱼ, j=1, . . . , D} is formed for the sub-space stretched on the full set of the calibration vectors, where Bᵢ are the vectors of the basis, and its dimension is D≦Nₐₗₗ. Then, each calibration measurement vector Vᵢ (from the set Vₐₗₗ) is represented by a row of its coordinates Uᵢ={Uᵢ₁, Uᵢ₂, . . . } in basis B so that

Vᵢ=ΣⱼUᵢⱼBⱼ. (11)

These coordinates are used to construct a covariance matrix of the calibration measurement vectors according to the expression:

Mᵥ=ΣᵢUᵢ^{T}Uᵢ, (12)

The eigenvalues eᵢ, i=1, . . . , D and eigenvectors Eᵢ, i=1, . . . , D of the covariance matrix Mᵥ are then determined. Next, the principal component analysis (PCA) invokes some principles, criteria or rules by which a part of the eigenvectors are selected to form the basis of a subspace on which further processing (such as least squares fitting) is performed. In one embodiment, a fixed small number of eigenvectors having the largest eigenvalues is selected. In another embodiment, the eigenvectors are selected from the comparison of their respective eigenvalues with eigenvalues that would be found if the calibration measurement vectors were replaced by pure noise measurement vectors obtained without actual samples placed in the measurement apparatus. In yet another embodiment, the principal component selection procedure can include analysis of variability of regression vectors as a function of the number of eigenvectors with the largest eigenvalues selected. The variability of a regression vector can be associated, for instance, with the norms of the derivatives of the regression functions defines above. In yet another embodiment, the principal component selection procedure can involve examination of errors of predicting constituent masses for a test set of measurements vectors as functions of the number of eigenvectors with the largest eigenvalues selected. In yet another embodiment, the principal component selection procedure can include analysis of the fractions of test measurement vectors obtained from target bodies, such as animals, which reside within the sub-space extended onto the eigenvectors selected as a function of the number of the largest eigenvalues selected. Some of these embodiments are explained in further detail below.

After the PCA, having selected the set of some Nₑ eigenvectors to be further used, a partial subspace, Sₚ is formed, of dimension Nₑ, stretched on these eigenvectors. Next, for each of the calibration measurement vectors in the Vₐₗₗ calibration measurement set, its projection P, onto the subspace Sₚ is determined. These projections, {Pᵢ, i=1, . . . ,Nₐₗₗ} are then used for subsequent partial least squares fitting, as follows. Let Aᵢ represent the mass of substance A in measurement i, then, using all coordinates with respect to the basis of the selected partial subspace, a linear system of equations is obtained:

Aᵢ=Pᵢ·R_{b}^{A}, i=1, . . . ,Nₐₗₗ (13)

where R_{b}^{A} are the unknown and sought-after components of the substance A regression vector with respect to the basis, Eᵢ, i=1, . . . ,Nₑ, of the partial subspace. The foregoing procedure of constructing the basis of the partial subspace assures that the number of eigenvectors is not larger than the number of vectors in the calibration measurement set (Nₑ≦Nₐₗₗ) so that the system of linear equations is either fully determined or over-determined. The system of linear equations can therefore be solved by a least squares fitting method, for example, as follows.

Let A represent a column of length Nₐₗₗ composed of the masses of substance A present in the Nₐₗₗ measurements, and let Pₐₗₗ represent the matrix of Nₐₗₗ rows, each of length Nₑ, formed by the Nₐₗₗ vectors Pᵢ. Then:

R_{b}^{A}=(Pₐₗₗ^{T}Pₐₗₗ)⁻¹Pₐₗₗ^{T}A. (14)

The components of the regression vector in the original basis are:

R_{A}=E^{T}R_{b}^{A}, (15)

where matrix E is formed by the rows made of the components of the partial subspace basis vectors.

In some “multi-sample” embodiments, one or more of the constituents have calibration measurement vectors obtained at more then one temperature. For such a constituent of a body, an evaluation of its temperature distribution can be made as follows. Instead of using a single regression vector for this constituent, separate regression vectors are calculated for each temperature of this constituent and these regression vectors are used to determine separately the masses of portions of this constituent at these temperatures in the body. The errors in the derived temperature distribution properties are smaller for constituents whose NMR properties change more widely with temperature. In particular, the fat tissue is most sensitive to temperature variations, so that, for instance, the canola oil equivalent temperature distribution can be better determined than that of lean tissue or water.

The results presented in FIG. 11 illustrate determination of temperature of fat using the technique described above, in comparison with temperature sensor data. The calibration measurement set used for the data presented in FIG. 11 includes measurements made on four samples of chicken breast meat, measurements made on two samples of saline solution, and measurements made on two samples of canola oil, each made at five different temperatures. The five calibration measurement temperatures are shown at 224. The testing was made on twenty five measurement vectors obtained from samples of canola oil held at eleven different temperatures, some of the testing temperatures outside the range of the five calibration temperatures, and some inside this range. The temperatures measured by sensors are shown at 225, and the temperatures predicted using the technique described above are shown at 226.

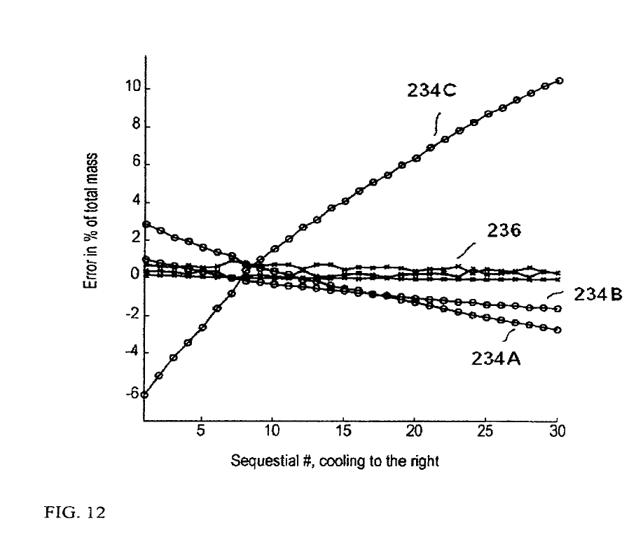

In some embodiments, the use of multi-temperature calibration measurements sets helps to reduce temperature-dependent errors in the determined constituent masses even when the details of the temperature distribution are not included in the body composition analysis requirements. FIG. 12 shows the evolution of errors in estimating the masses of fat, lean, and saline in a cooling test sample. Each sequential measurement indicated on the ordinate axis of the graph in FIG. 12 corresponds to a lower temperature of the sample which was initially heated to about 38 degrees C. and was then allowed to cool to nearly the room temperature (that is the temperature decreases from left to right). Dotted lines 234A, 234B and 234C (for fat, lean, and saline respectively) represent errors corresponding to single-temperature calibration, while solid lines 236 represent multi-temperature calibration. As can be inferred from FIG. 12, using multiple samples of each constituent in the calibration measurement set at a plurality of temperatures reduces the analysis error where the sample is subject to variable or unknown temperatures.

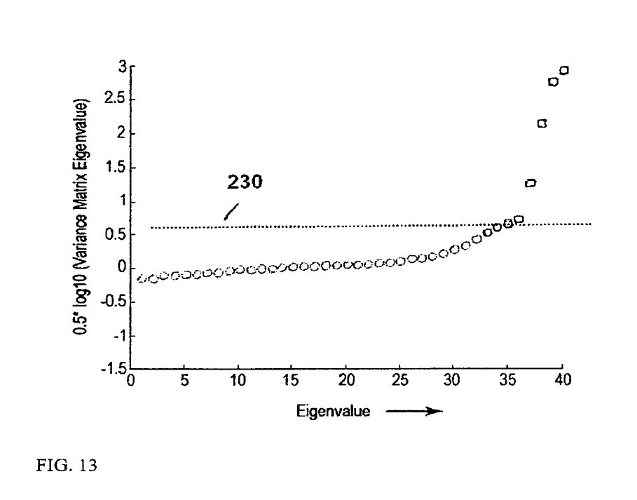

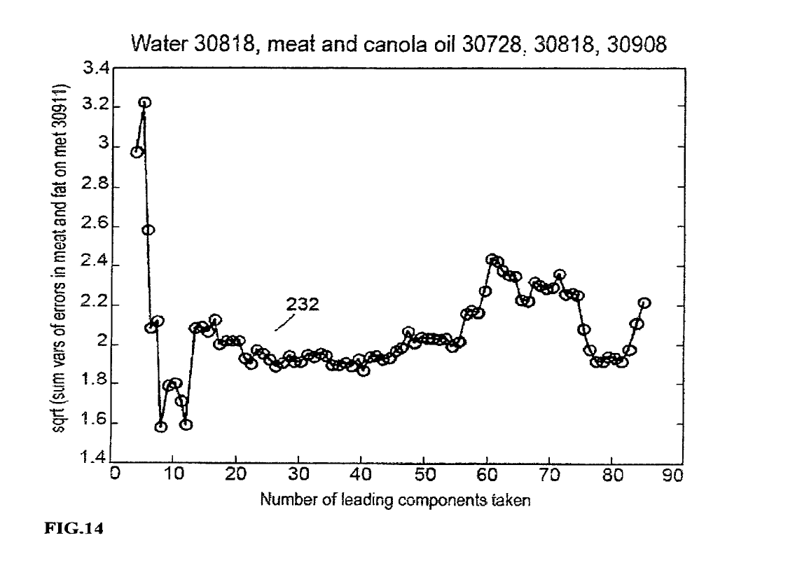

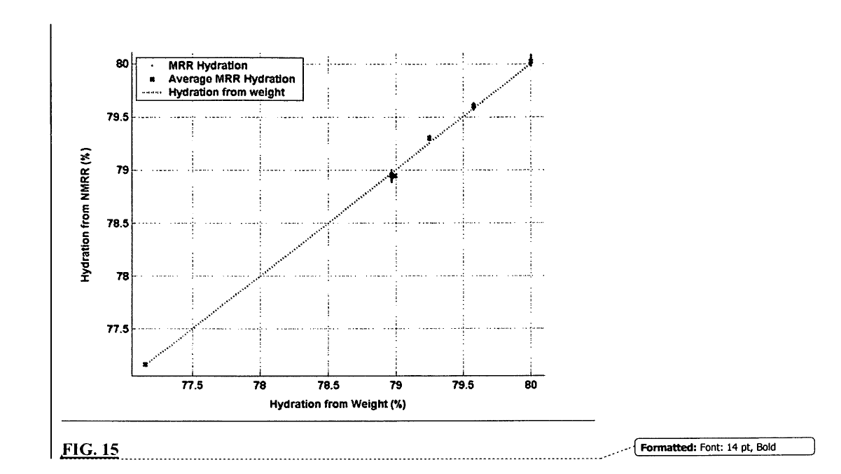

FIGS. 13 and 14 illustrate two of the procedures described above for selecting the number, Nₑ, of eigenvectors with the largest eigenvalues, that are to be used for the partial least squares fitting to generate the regression vectors for each constituent. The graph in FIG. 13 shows the variance matrix eigenvector number on the coordinate axis and the variance matrix eigenvalues for each corresponding eigenvector on the ordinate axis. The procedure illustrated in FIG. 13 is based on a comparison of the variance matrix eigenvalues with the maximum eigenvalue of noise in the acquisition system. Acquisition system noise eigenvalues can be determined from data acquired without a sample in the chamber (18 in FIG. 1). The noise level is shown by the dashed line 230 in FIG. 13. In one embodiment, only eigenvectors with eigenvalues exceeding the maximum eigenvalue of the acquisition system noise are taken to form the partial subspace, Sₚ.

FIG. 14 illustrates a different procedure for selecting the most significant eigenvectors. The graph in FIG. 14 shows the sum of the squares of the analysis errors for some test measurements, shown at curve 232, plotted with respect to the number of calibration sample measurement vectors used to generate the regression vector. FIG. 14 suggests that there is an optimum number of calibration measurement vectors that should be used to generate the regression vectors for the composition analysis procedure of the invention. In the embodiment illustrated in FIG. 14, determining the set of significant eigenvectors is based on the errors of mass predictions for a set of test measurements.

The procedures representing different embodiments of the present invention have as a goal better accuracy and precision in analyzing body composition in the presence of different uncertainty factors such as natural variations of NMR relaxation properties of the same substance present in the body, or uncertainty due to variations in temperature of a constituent (for example, possible variation of temperature of fat tissue depending on its location within the body being analyzed).

Methods according to the invention include a number of specific applications. In one implementation, the effects of certain medications intended to affect body fat content may be evaluated. A human patient, or laboratory test animal may be initially analyzed with respect to total fat content, total lean body mass content and/or total free water content using a method and apparatus as explained above. A medication intended to affect total body fat content may be administered to the human patient or test animal. After a selected time, the human patient or laboratory test animal may again be evaluated as to fat content, lean mass content and/or free water content using a method and apparatus as explained above.

In some implementations, it is possible to estimate bone mass of the patient or animal by subtracting the fat content, lean mass content and free water content determined using methods and apparatus explained above from the total body mass (or weight). Such bone mass evaluation may have application in evaluation of medications used to treat bone loss. For example, bone mass may be estimated as explained above, and a medication intended to affect bone mass may be administered to the patient or test animal. After a selected time, the patient or animal may have its bone mass estimated again using the technique explained above.

In some embodiments, the amount of free water can be measured by means of a separate CPMG measurement sequence lasting significantly longer than the typical relaxation times of fat and lean tissue. Such a measurement sequence should be preceded by a sufficiently long recovery period to enable the free water to reach magnetization equilibrium. The amount of free water is determined from the signal at the tail part of the measurement sequence.

In some embodiments, such a separate CPMG sequence, preceded by a sufficiently long recovery period to enable the free water to reach magnetization equilibrium, can also be used to determine the amount of total water, which includes the free water and the water contained in the muscle tissue. Such a procedure may be performed as follows.

A measurement subsequence, which can be the whole sequence or at least a part of the sequence of received spin echo magnitudes is extrapolated back to the time of the excitation RF pulse. The resulting zero time crossing value is proportional to the total amount of protons in the material being analyzed. These protons are comprised of protons contained in fat and protons contained in water. The amount of the former can be found from the amount of fat, determined as explained above using the fat regression vector. The total water is determined from the total amount of protons in water calculated as the difference between the total number of protons and the protons disposed in fat.

In some embodiments, hydration of lean tissue can be found by yet another method, fully based on the use of regression vectors, and fully analogous to the method for determining the temperature of fat described above. The regression vectors are determined from training samples of lean tissue with known amounts of hydration, and then used for calculating the average hydration of a sample having unknown hydration. In some embodiments, the hydration of training samples can be determined by a combination of drying some samples, such as by heating, and drying similar samples by freezing, the similarity being established by thorough mixing of the lean tissue.

Examples of comparison of hydration values obtained as explained above and values obtained from comparing weights of the same sample after freeze-drying are shown in FIG. 15.

In any of the foregoing evaluation techniques for various medications, it is possible, using methods and apparatus according to the invention, to evaluate the efficacy of the medication, and whether the treatment afforded by the particular medication requires alteration, for example, in the composition of the medication or the dosage thereof, or whether a physical therapy regimen may be altered or amended. Accordingly, in some implementations, after a selected time, a patient or test animal may have fat content, lean mass and/or free water content evaluated using a method and apparatus according to the invention. A dosage or composition of a medication may be changed, or a physical therapy, such as a particular exercise regimen may be altered. After such alteration or change, and after a selected time, the patient or animal may be again analyzed using a method and apparatus according to the invention. The efficacy of the amended or changed treatment may be monitored using the foregoing technique at selected times.

A particular advantage of an apparatus and methods according to the invention is that they may be used to obtain accurate measurements even on patients who are unable or unwilling to remain completely still during the measurement procedure. The present invention is therefore believed to have particular application on human infants and children.

While the invention has been described with respect to a limited number of embodiments, those skilled in the art, having benefit of this disclosure, will appreciate that other embodiments can be devised which do not depart from the scope of the invention as disclosed herein. Accordingly, the scope of the invention should be limited only by the attached claims.

What is claimed is:

1. A method for analyzing composition of a human body, comprising:
inducing a substantially homogeneous static magnetic field in the entire body;
inducing a substantially homogeneous radio frequency magnetic field in the entire body so as to induce nuclear magnetic resonance effects in the body; and
analyzing nuclear magnetic resonance signals emanating from the entire body.

2. The method of claim 1 wherein the analyzing comprises:
composing at least a part of the nuclear magnetic resonance signals into a measurement vector;
calculating mass of at least one constituent as a predetermined function of the measurement vector, the predetermined function representing the at least one constituent and defining a standard for a range of compositional and/or temperature variations of the at least one constituent.

3. The method of claim 1 further comprising determining at least one of fat content, lean content and free water content from the nuclear magnetic resonance signals.

4. The method of claim 3 further comprising:
administering a treatment to the body;
repeating the inducing the static and radio frequency magnetic fields, analyzing the measurements and determining at least one of fat content, lean mass content and free water content; and
evaluating the efficacy of the treatment from the repeated determination of the at least one of fat content, lean mass content and free water content.

5. The method of claim 4 further comprising:
adjusting the treatment;
administering the adjusted treatment;
repeating the inducing the static and radio frequency magnetic fields, analyzing the measurements and determining at least one of fat content, lean mass content and free water content; and
evaluating the efficacy of the adjusted treatment from the repeated determination of the at least one of fat content, lean mass content and free water content.

6. The method of claim 4 wherein the treatment comprises a medication.

7. The method of claim 4 wherein the treatment comprises a physical therapy regime.

8. The method of claim 3 further comprising determining bone mass from the analyzed nuclear magnetic resonance measurements.

9. The method of claim 8 further comprising:
administering a bone mass treatment to the body;
repeating the inducing the static and radio frequency magnetic fields, analyzing the measurements and determining the bone mass and
evaluating the efficacy of the bone mass treatment from the repeated determination of the bone mass.

10. The method of claim 8 further comprising:
adjusting the bone mass treatment;
administering the adjusted treatment;
repeating the inducing the static and radio frequency magnetic fields, analyzing the measurements and determining the bone mass; and
evaluating the efficacy of the adjusted bone mass treatment from the repeated determination of the bone mass.

11. The method of claim 1 further comprising adjusting the magnitude of the static magnetic field while keeping the radio frequency fixed, in order to optimize the magnetic resonance signal and minimize distortions therein caused by residual inhomogeneities in the static magnetic field.

12. The method of claim 1 further comprising adjusting the frequency of the radio frequency magnetic field by an amount inversely related to a delay time between refocusing pulses, and repeating the inducing the radio frequency magnetic field and analyzing the nuclear magnetic resonance signals, in order to optimize the magnetic resonance signal and minimize its distortions caused by residual inhomogeneities in the static magnetic field.

13. The method of claim 1 wherein the inducing a substantially homogeneous radio frequency magnetic field in the body comprises generating a predetermined series of pulses each having a plurality of sequences of pulses wherein sequence durations and intervals between sequences are selected so as to optimally measure transverse and longitudinal relaxation rates of at least one constituent of the body.

14. The method of claim 13 wherein a spin echo induced by each of the pulses contributes to a single component of a measurement vector calculated as a convolution of the spin echo amplitude with at least one of a kernel and a filter.

15. The method of claim 14 wherein the at least one of a kernel and a filter is selected to reduce effects of residual inhomogeneities of the static magnetic field in the volume of the body.

16. The method of claim 13 wherein the sequences comprise at least one Carr-Purcell-Meiboom-Gill sequence.

17. The method of claim 2 wherein the predetermined function is determined from calibration measurement vectors composed from nuclear magnetic resonance signals obtained from calibration samples of known composition.

18. The method of claim 17 wherein at least one of the calibration samples represents at least one constituent of the body.

19. The method of claim 18 wherein the calibration samples include at least two samples representing different composition variations of the at least one constituent.

20. The method of claim 19 further comprising determining a spatial distribution of compositional variations of at least one constituent from the nuclear magnetic resonance signals.

21. The method of claim 18 wherein the calibration samples include at least two samples representing the at least one constituent at different temperatures.

22. The method of claim 21 further comprising at least one of determining average temperature of the at least one constituent from the nuclear magnetic resonance signals and determining a temperature distribution of the at least one constituent from the nuclear magnetic resonance signals.

23. The method of claim 18 wherein the calibration samples include at least two samples representing the at least one constituent at different hydrations.

24. The method of claim 23 further comprising at least one of determining average hydration of the at least one constituent from the nuclear magnetic resonance measurements and determining a hydration distribution of the at least one constituent from the nuclear magnetic resonance measurements.

25. The method of claim 18 wherein at least one calibration sample representing fat tissue in the body comprises vegetable oil.

26. The method of claim 18 wherein at least one calibration sample representing lean body tissue comprises pig muscle tissue.

27. The method of claim 26 wherein intrinsic compositional variation of samples of the pig muscle tissue is compensated by using a plurality of pig muscle tissue calibration samples.

28. The method of claim 18 wherein at least one calibration sample representing free water comprises saline solution.

29. The method of claim 2 wherein the predetermined function of the measurement vector is linear, whereby the mass of the at least one constituent is determined as a scalar product of the measurement vector and a predetermined regression vector.

30. The method of claim 29 further comprising determining a set of regression vectors, each regression vector in the set corresponding to a different constituent of the body by approximating an arbitrary measurement vector as a linear combination of predetermined base vectors.

31. The method of claim 30 wherein at least one of the predetermined base vectors represents a single constituent of the body.

32. The method of claim 30 wherein the predetermined regression vector for each of the constituents is derived from calibration measurement vectors composed from nuclear magnetic resonance signals obtained on calibration samples of known composition.

33. The method of claim 30 wherein the determining of the regression vectors for each of the constituents comprises:
performing principal component analysis on a set of calibration measurement vectors;
selecting a set of significant principal components;
performing a partial least square fitting of the set of calibration measurement vectors by linear combinations of the selected set of significant principal components; and
calculating the regression vectors.

34. The method of claim 33 wherein the determining the regression vectors for each of the constituents comprises normalization of each calibration measurement vector to the mass of the sample used to make the calibration measurements

35. The method of claim 33 wherein the selecting the set of significant principal components comprises comparing eigenvalues of a covariance matrix of calibration measurements with eigenvalues of a covariance matrix of noise data.

36. The method of claim 33 wherein the selecting the set of significant principal components comprises determining errors of mass predictions for a set of test measurements.

37. The method of claim 33 wherein the set of significant principal components is selected by determining a degree to which measurement vectors of a test set of body parts are encompassed by a sub-space of the selected principal components.

38. The method of claim 33 wherein the set of significant principal components is selected by determining variability of the regression vector, regarding the components of a regression vector as values of a function of the component's sequential number and calculating the norm of the derivative of the regression function.

39. The method of claim 33 wherein the determining the regression vectors is preceded by smoothing of calibration measurement vectors, wherein the value of a vector component is set to be a piece-wise smooth function of the vector component number.

40. The method of claim 1 wherein durations of sequences of measurements made of the nuclear magnetic resonance signals are sufficiently long for contributions to the nuclear magnetic resonance signals from fat tissue in the body and lean tissue in the body to decay to substantially zero amplitude, such that the remaining nuclear magnetic resonance signals are substantially only from free water in the body and such remaining signals are used to determine an amount of the free water in the body.

41. The method of claim 1 wherein an observed decay of the nuclear magnetic resonance signals in measurement sequences is extrapolated back to an initial excitation time, and an amplitude of the extrapolation is used to determine a total amount of water in the body comprising free water and water in the lean tissue in the body.

42. An apparatus for analyzing composition of a human body, comprising:
a magnet for inducing a substantially homogeneous static magnetic field in a chamber having a volume at least as large as an entire human body;
means for inducing a substantially homogeneous, pulsed radio frequency magnetic field in the entire human body; and
means for analyzing nuclear magnetic resonance signals from the entire body induced therein by the static magnetic field and the radio frequency magnetic field.

43. The apparatus of claim 42 wherein the magnet comprises a plurality of wound coil electromagnets, each having a controllable electric current source operatively connected thereto such that a spatial distribution of the static magnetic field is controllable. The apparatus of claim 43 wherein the magnet comprises orthogonally arranged shims having thickness selected such that the static magnetic field is substantially homogeneous within the entire chamber.

44. The apparatus of claim 44 further comprising a pole piece disposed at each longitudinal end of each of the coils.

45. The apparatus of claim 42 wherein the wound coil electromagnets are configured to shield the radio frequency magnetic field from leaving a predefined chamber. The apparatus of claim 42 further comprising an active radio frequency spoiler to substantially neutralize any radio frequency energy from radiating outside a defined volume.

46. The apparatus of claim 42 wherein the means for inducing a pulsed radio frequency magnetic field comprises an antenna wound such that its spatial distribution of sensitivity is substantially homogeneous within the chamber. The apparatus of claim 48 where the antenna comprises two contra wound, center-tapped series connected coils such that an inductance of the coils is substantially reduced.

47. The apparatus of claim 48 wherein the antenna comprises higher current density at longitudinal ends thereof than in a longitudinal center thereof.

48. The apparatus of claim 42 wherein the means for inducing a pulsed radio frequency magnetic field comprises means for adjusting a pulse width of radio frequency current pulses to as to increase a bandwidth of the radio frequency magnetic field.

49. The apparatus of claim 42 wherein the means for inducing a pulsed radio frequency magnetic field comprises means for adjusting a frequency of radio frequency current such that residual inhomogeneities in the static magnetic field are compensable.

50. The apparatus of claim 42 wherein the means for analyzing comprises a correction filter in a receiver circuit configured to compensate for the residual inhomogeneities in the static magnetic field.

51. The apparatus of claim 42 wherein a frequency of the radio frequency magnetic field is at most about 500 kilohertz.

52. The apparatus of claim 42 wherein the means for analyzing comprises means for determining at least one of mass of free water, mass of fat and mass of lean tissue in the body from the nuclear magnetic resonance signals.

53. A method for investigating a property of a body, comprising:
inducing a time varying electromagnetic field in the body; and
measuring an effect induced by the time varying electromagnetic field in the body, wherein a frequency of the time varying electromagnetic field is selected such that an attenuation of the field in the body is at most about equal to a precision with which the property is determined.

54. The method of claim 53 wherein the effect comprises nuclear magnetic resonance spin echo amplitude, the body is a human subject, and the frequency is at most about 500 kilohertz.
